(19) United States
(12) Patent Application Publication
(10) Pub. No.: US 2025/0045963 A1
(43) Pub. Date: Feb. 6, 2025

(54) IMAGE PROCESSING APPARATUS, TERMINAL, AND MONITORING METHOD

(71) Applicant: Morpho, Inc., Tokyo (JP)

(72) Inventors: Miyuki Kashiwagi, Tokyo (JP); Shun Hirai, Tokyo (JP)

(73) Assignee: Morpho, Inc., Tokyo (JP)

(21) Appl. No.: 18/719,259

(22) PCT Filed: Dec. 13, 2022
(86) PCT No.: PCT/JP2022/045920

§ 371 Date: Jun. 13, 2024

(30) Foreign Application Priority Data
Dec. 17, 2021 (JP) 2021-205527
Nov. 30, 2022 (JP) 2022-191870

(51) Int. Cl. G06T 7/90; G06V 10/56

(52) U.S. Cl. CPC G06T 7/90; G06V 10/56; G06T 2207/10024; G06T 2207/30201

(57) ABSTRACT

The present invention enables output of information based on color information. A calculation unit and an output unit are included, the calculation unit being configured to calculate relative relationship information on a difference between first color information in a first region included in a captured image and second color information in a second region included in the captured image, and the output unit being configured to output time-series relative relationship information of a plurality of captured images captured at different points in time.

### TECHNICAL FIELD

The present disclosure relates to an image processing apparatus, a terminal, and a monitoring method.

### BACKGROUND ART

For example, image processing apparatuses have been proposed that perform analyses based on color information in an image in which the subject is a human, thereby determining the skin condition, skin age, and the like of that human. For example, Patent Document 1 discloses an image processing apparatus that instructs the photographer to correct the position or orientation of the image capturing unit according to whether or not the face region is in the target region within the shooting region as a result of face region detection.

### CITATION LIST

### Patent Document

Patent Document 1: JP 2008-118276A

### SUMMARY OF INVENTION

According to an aspect of the present disclosure, included are: a calculation unit configured to calculate relative relationship information on a difference between first color information in a first region included in a captured image and second color information in a second region included in the captured image; and an output unit configured to output time-series relative relationship information of a plurality of captured images captured at different points in time.

### BRIEF DESCRIPTION OF DRAWINGS

FIG. 1 is a diagram showing an example of the functional configuration of an image processing apparatus.

FIG. 2 is a diagram showing an example of a time-series change in color difference.

FIG. 3 is a flowchart showing an example of the flow of image processing (color information processing).

FIG. 4 is a diagram showing an example of the functional configuration of a terminal according to an example.

FIG. 5 is a flowchart showing an example of the flow of processing that is performed by the terminal according to an example.

FIG. 6 is a diagram showing another example of the functional configuration of the terminal according to an example.

FIG. 7 is a diagram showing an example of the functional configuration of a server according to an example.

FIG. 8 is a flowchart showing an example of the flow of processing that is performed by the terminal and the server according to an example.

FIG. 9 is a diagram showing an example of a display screen displayed on a display unit of the terminal according to an example.

FIG. 10 is a diagram showing an example of a display screen displayed on the display unit of the terminal according to an example.

FIG. 11 is a diagram showing an example of a display screen displayed on the display unit of the terminal according to an example.

FIG. 12 is a diagram showing an example of a display screen displayed on the display unit of the terminal according to an example.

FIG. 13 is a diagram showing an example of a display screen displayed on the display unit of the terminal according to an example.

FIG. 14 is a diagram showing an example of a display screen displayed on the display unit of the terminal according to an example.

FIG. 15 is a diagram showing an example of a display screen displayed on the display unit of the terminal according to an example.

FIG. 16 is a diagram showing an example of a display screen displayed on the display unit of the terminal according to an example.

FIG. 17 is a diagram showing an example of a recording medium.

FIG. 18 is a diagram showing that time-series data of the fatigue level is displayed on the terminal.

FIG. 19 is a diagram showing a configuration example of a terminal.

FIG. 20 is a diagram showing a configuration example of another terminal.

### DESCRIPTION OF EMBODIMENTS

An example of an embodiment for carrying out the present disclosure will be described below with reference to the drawings. In the following description, the same constituent elements are denoted by the same reference numerals, and redundant descriptions thereof have been omitted. Note that the constituent elements described in the following embodiments are merely examples, and are not intended to limit the scope of the disclosure thereto.

### EMBODIMENT

Hereinafter, an example of an embodiment for realizing an image processing technique according to the present disclosure will be described.

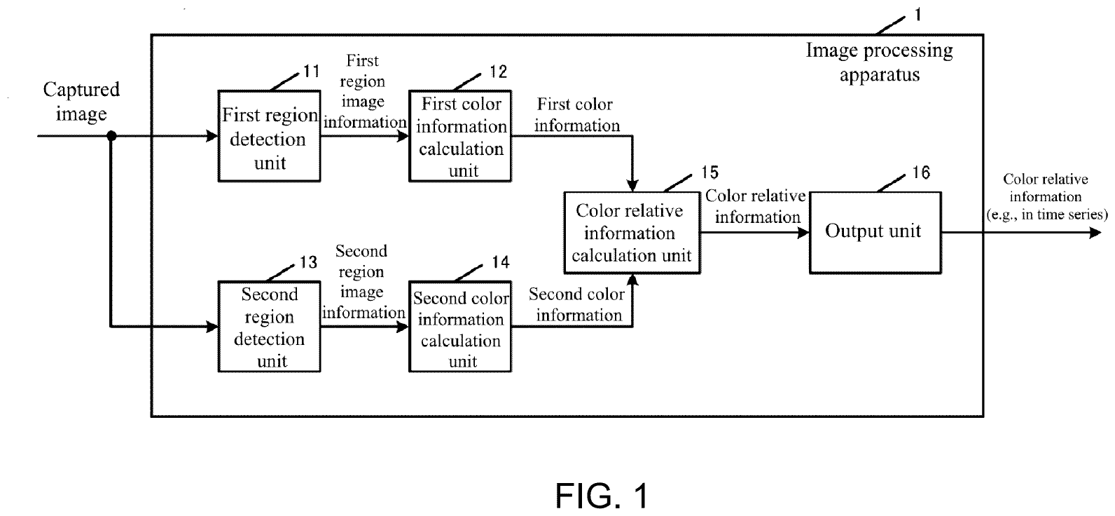

FIG. 1 is a block diagram showing an example of the functional configuration of an image processing apparatus 1 according to an aspect of this embodiment.

The image processing apparatus 1 may include, for example, a first region detection unit 11, a first color information calculation unit 12, a second region detection unit 13, a second color information calculation unit 14, a color relative relationship information calculation unit 15, and an output unit 16. These units are, for example, functional units (functional blocks) included in unshown processing units (processing devices) or control units (control devices) of the image processing apparatus 1, and can include a processor such as a CPU or DSP or an integrated circuit such as an ASIC.

The first region detection unit 11 has a function of detecting a first region in a captured image captured by an unshown image capturing unit, for example, based on the captured image.

The captured image may be a captured image captured by any camera.

The details will be described later in the example, but the captured image may be, for example, an image captured by a front camera.

The front camera may be a camera in which an unshown display unit can display a photographer when the photographer captures an image, specifically, for example, a camera (in-camera) installed on the front side (the side with a face having the display unit) of the device body. The captured image may be a captured image captured by a rear camera.

The rear camera may be a camera in which an unshown display unit cannot display a photographer when the photographer captures an image, specifically, for example, a camera (back camera, out-camera) installed on the rear side (opposite the side where the display unit is formed) of the device body.

The first color information calculation unit 12 has a function of calculating color information, for example, expressed in hue, saturation, and lightness defined in the HSL color space, for example, based on image information in the first region detected by the first region detection unit 11.

The second region detection unit 13 has a function of detecting a second region in the above-mentioned captured image, for example, based on the captured image.

The second color information calculation unit 14 has a function of calculating color information, for example, defined in the above-mentioned HSL color space, for example, based on image information in the second region detected by the second region detection unit 13.

The first region detection unit 11 can detect a first region from a captured image, the first region being, for example, a region set by a user in advance or defined by a program. The same applies to the second region detection unit 13.

Specific examples of the first region and the second region will be described later.

The color relative relationship information calculation unit 15 has a function of, for example, based on the first color information calculated by the first color information calculation unit 12 and the second color information calculated by the second color information calculation unit 14, calculating color relative relationship information, which is information on their relative relationship.

The color relative relationship information may include, for example, a color difference, which is the distance between colors based on the first color information and the second color information. The color difference is a scalar value and can be calculated, for example, as a Euclidean distance from the first color information and the second color information.

The color relative relationship information may contain, in addition to or instead of the color difference, a color relative relationship vector expressed as a vector. The color relative relationship vector can be calculated, for example, as a difference vector between first color information in three dimensions (e.g., components of the HSL color space mentioned above) and second color information in three dimensions.

The output unit 16 has a function of outputting the color relative relationship information calculated by the color relative relationship information calculation unit 15.

Although not shown, it is also possible that a memory in which the color relative relationship information calculated by the color relative relationship information calculation unit 15 is accumulated and stored is provided inside or outside the image processing apparatus,
wherein the color relative relationship information is stored in the memory, for example, in time series. In this case, the output unit 16 may output, for example, the relative relationship information stored in the memory for a plurality of captured images captured at different points in time, in time series.

The first region detection unit 11 and the second region detection unit 13 may be configured as a single region detection unit having the same function (a function of, in response to input of an image, outputting an image in which a specific region in the image is detected (extracted)).

Furthermore, the first color information calculation unit 12 and the second color information calculation unit 14 may be configured as a single color information calculation unit having the same function (a function of outputting color information in response to input of an image).

### Principle

The first region and the second region may be, for example, two different regions included in the captured image.

Also, as just an example, the first region and the second region may be, for example, different regions in the same subject included in the captured image (different regions included in the same subject).

Since the circumstances and environments in which an image is captured can vary from time to time, it is difficult to identify the color of a subject from the captured image due to the influence of light and other factors, even when the same subject is captured. That is to say, if there is no object in the captured image whose color is known to the image processing apparatus, it is difficult to identify the color of a subject from the captured image because there is no color that can be used as a comparison target (standard). Although a color patch or color chart would be helpful, it is cumbersome for the user.

Therefore, instead of identifying the color of a subject, this embodiment calculates, for each captured image, relative relationship information (color relative relationship information) on a relative relationship between the first color information in the first region and the second color information in the second region of the same subject included in the captured image. It is possible to analyze how much the color of the first region deviates from the color of the second region, from the calculated relative relationship information.

It can be assumed that the influences of light due to the image capture environment and the like are the same or similar in the same subject. Therefore, the color relative relationship information may also be said to be information that is not affected or is unlikely to be affected by the image capture environment and the like.

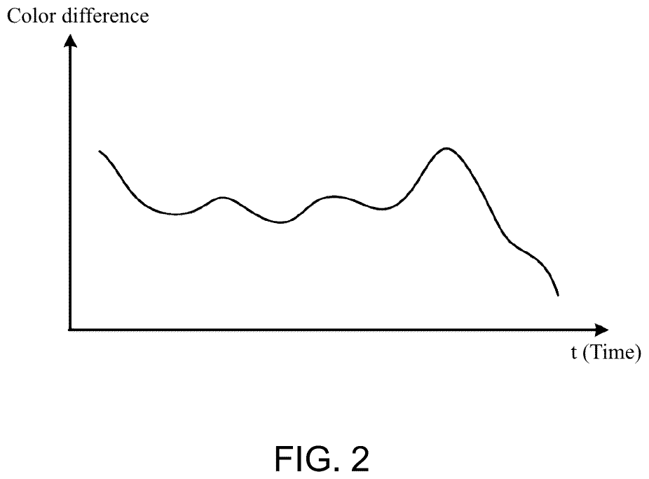

FIG. 2 is a diagram showing a time-series change in color difference in the case in which the color difference is used as color relative relationship information output from the output unit 16, in which the horizontal axis indicates the time (t) and the vertical axis indicates the color difference. In this example, the color difference is shown as a graph of continuous curves.

The closer the color difference approaches zero, the closer the color of the first region is to the color of the second region. Conversely, the further away from zero the color difference is, the more the color of the first region deviates from the color of the second region. This application will be briefly described below and will be discussed in detail in “Examples” below.

The subject may be an animal, including, for example, a human (a human being, a figure, or a person) (note that a human is included in an animal in this example).

In this example, the case in which a human is taken as a subject will be described as an example. In this case, the first region and the second region may be, for example, different regions in the same human included in the captured image.

In this case, the first region may be, for example, either or both of the following regions:
Face region; and
Neck region.

Furthermore, the second region may be, for example,
Inner region of arm.

The “arm” is defined as the part from one's shoulder to one's wrist.

Furthermore, the face region be, for example,
Cheek region.

Furthermore, the inner region of an arm may be, for example, either or both of the following regions:
Inner region of upper arm; and
Inner region of wrist.

Furthermore, a combination the first region and the second region may be a combination of any of those listed above.

In this specification, for example, based on medical definitions, the part that is closer to a human shoulder than the elbow is will be referred to as an “upper arm”, and the part that is closer to a human hand than the elbow is will be referred to as a “forearm”.

The inner region of an arm is used as an example because the inner side of an arm is considered to be less prone to sunburn (less likely to change skin color). However, even though it is the inner side of an arm, the inner side of the forearm may also be sunburned when a person is wearing short-sleeved clothing. For this reason, the inner side of an upper arm may be used as the inner side of the arm. In addition, since a wrist may be less prone to sunburn, the inner side of the wrist may be used even if it is the forearm.

The inner side of an upper arm and the inner side of a wrist are considered to be particularly less prone to sunburn and to maintain its skin color. Therefore, as will be described in more detail in “Examples” below, the color of the inner side of an upper arm or the inner side of a wrist can be used as an ideal skin color (skin color before being affected by external disturbances such as sunlight), and how much the color of the face (cheek) and neck, which are likely to be affected by sunlight, deviates from the color of the inner side of the upper arm or the inner side of the wrist can be analyzed using the above-described color relative relationship information.

With the recent trend of beauty cosmetic sales, for example, not only the region of a human face (cheek), but also the region of a human neck may be considered as a target region for anti-aging and other skin beautifying products.

Note that the face region may be regions other than cheeks.

The subject is not limited to a human, and may be animals other than humans.

The subject is not limited to an animal, and may be objects.

Furthermore, the object regions are not limited to the first region and the second region that are different regions in the same subject included in a captured image, and, for example, the first region and the second region that are any different regions included in a captured image may be taken as object regions to calculate color relative relationship information, and how much the colors deviate from each other may be analyzed. For example, how much the colors deviate from each other may be analyzed while taking one region of one subject as the first region and one region of another subject as the second region.

### Image Processing Procedures

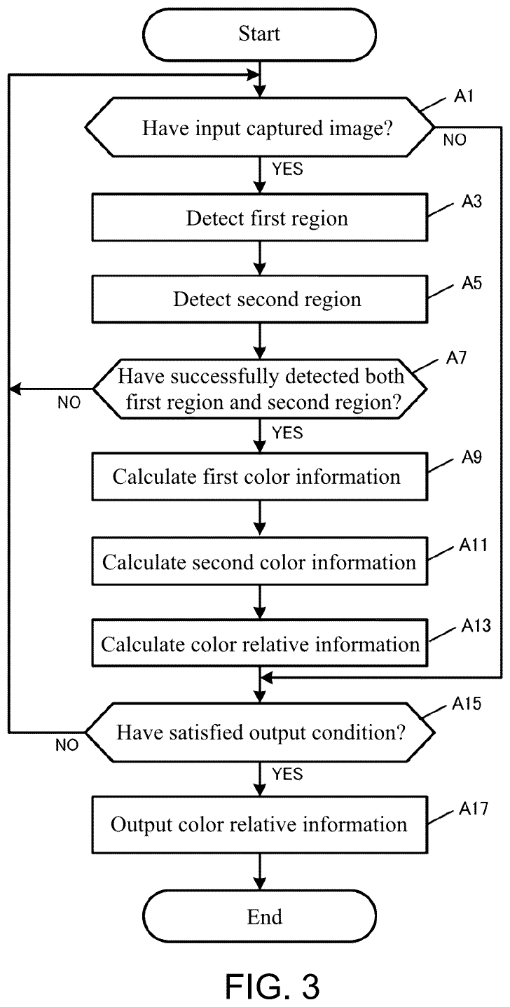

FIG. 3 is a flowchart showing a procedure example of image processing in this embodiment.

The processing shown in the flowchart in FIG. 3 is realized by, for example, an unshown processing unit of the image processing apparatus 1 reading and executing the code of an image processing program stored in an unshown storage unit in an unshown RAM or the like.

Although FIG. 1 does not show any storage unit as a constituent element of the image processing apparatus 1, storage units may be included in the constituent elements of the image processing apparatus 1.

The flowcharts described below are merely an example of the image processing procedure in this embodiment, and other steps may be added or some steps may be deleted.

First, the image processing apparatus 1 determines whether or not a captured image has been input (A1). If it is determined that a captured image has been input (A1: YES), the first region detection unit 11 performs processing for detecting a first region from the input captured image (A3). In this processing, the first region detection unit 11 detects a first region, for example, through region extraction processing.

In the region extraction processing, for example, a first region can be detected using the Key Point Detection method. Semantic segmentation may also be performed on the captured image using a deep learning method such as FCN (Fully Convolutional Network), SegNet, or U-Net. The position of pixels classified as the first region and the color information on the pixels are then obtained as the extraction results.

For example, if the first region is a cheek region, the position of pixels classified as the cheek region and the color information on the pixels are obtained as the extraction results.

It is also possible to extract features from the captured image using, for example, HOG (Histogram of Oriented Gradients) feature amounts and extract the first region using a discriminator such as SVM (Support Vector Machine).

If the first region is the skin, the color histogram can be used to evaluate the skin tone-like quality of each pixel, and the first region can be extracted from the captured image. The skin tone-like quality may be evaluated for the SVM extraction results.

The results from deep learning and these results may also be combined for region extraction.

In a similar manner, the second region detection unit 13 detects a second region from the captured image (A5). This detection can also be realized, for example, through the above-described region extraction processing. For example, if the second region is an inner region of an upper arm or an inner region of a wrist, the position of pixels classified as such a region and the color information on the pixels are obtained as the extraction results through a method such as the Key Point Detection method or the semantic segmentation method.

The image processing apparatus 1 may, for example, perform the extraction of the first region and the extraction of the second region at once based on the results of semantic segmentation.

Next, the image processing apparatus 1 determines whether or not the first region and the second region have been successfully detected (A7). This determination can be realized, for example, by determining whether or not the first region occupies a set percentage of the captured image based on the detection result (extraction result) of A3 and determining whether or not the second region occupies a set percentage of the captured image based on the detection result (extraction result) of A5.

The determination in A7 may be performed by determining whether or not the first and second regions occupy a set percentage of the captured image.

If it is determined that at least one of the detections failed (A7: NO), the image processing apparatus 1 returns the processing to A1.

In this case, the image processing apparatus 1 may perform some error processing and then return the processing to A1. Since there may be an issue with the image capture, information may be output to alert the user to this fact, for example.

If the first region and the second region have been successfully detected (A7: YES), the first color information calculation unit 12 calculates first color information based on the detection result of A3 (A9). For example, the average value of the color information of pixels is calculated for each image patch (small region of an image) of a set size. Then, based on the average value, for example, the color information (first color information) is calculated for each image patch in a predetermined color space.

In a similar manner, the second color information calculation unit 14 calculates second color information based on the detection result of A5 (A11). This calculation can be performed as in A9, for example.

Next, the color relative relationship information calculation unit 15 calculates color relative relationship information based on the first color information calculated in A9 and the second color information calculated in A11 (A13).

For example, if a color difference is to be calculated as the color relative relationship information, the color relative relationship information calculation unit 15 calculates a color difference by calculating the Euclidean distance between the first color information calculated in A9 and the second color information calculated in A11 or the like.

For example, if a color relative relationship vector is to be calculated as the color relative relationship information, the color relative relationship information calculation unit 15 calculates a vector (in three dimensions) of the difference between the three-dimensional color information calculated in A9 and the three-dimensional color information calculated in A11.

Next, the image processing apparatus 1 determines whether or not the output condition of the color relative relationship information is satisfied (A15). If it is determined that the output condition is not satisfied (A15: NO), the image processing apparatus 1 returns the processing to A1. On the other hand, if it is determined that the output condition is satisfied (A15: YES), the image processing apparatus 1 ends the processing.

As the output condition, for example, various conditions such as that output is performed for each captured image, that color relative relationship information has been calculated for a set number of captured images, or that color relative relationship information has been calculated for a set period of captured images can be specified.

In the case of calculating color relative relationship information for a set number of captured images, the image processing apparatus 1 may output, for example, the average value, the median value, or the like of the color relative relationship information calculated for the respective captured images. The same applies to the case of calculating color relative relationship information for a set period of captured images. That is to say, the image processing apparatus 1 may output one piece of color relative relationship information from a plurality of captured images. The outputting one piece of color relative relationship information from a plurality of captured images is expected to further reduce the influence of the image capture environment.

In this processing, Step A5 is performed after Step A3 but this order may be reversed. The same applies to Steps A9 and A11.

### EXAMPLES

### (1) Example of Terminal

Hereinafter, an example of a terminal to which the above-described image processing apparatus 1 is applied or that includes the above-described image processing apparatus 1 will be described. The terminal may be, for example, a terminal device that a user possesses, such as a cell phone including a smartphone, a camera, a PDA, a personal computer, a navigation device, a wristwatch, and various tablet terminals.

As an example, the implementation of a smartphone, which is a type of cell phone with a camera function (with an image capturing function) will be described. In the description, it is illustrated and described as a terminal 100.

Note that examples to which the present disclosure can be applied are not limited to the examples described below.

### Functional Configuration

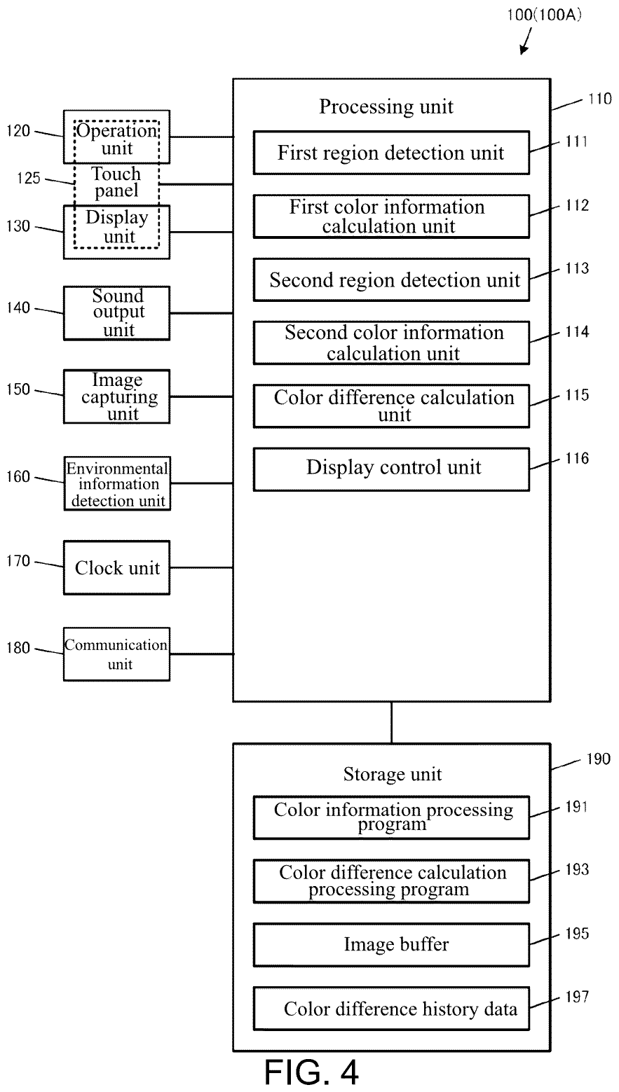

FIG. 4 is a diagram showing an example of the functional configuration of a terminal 100A, which is an example of the terminal 100 that is a smartphone in this example.

The terminal 100 includes, for example, a processing unit 110, an operation unit 120, a touch panel 125, a display unit 130, a sound output unit 140, an image capturing unit 150, an environmental information detection unit 160, a clock unit 170, a communication unit 180, and a storage unit 190.

The processing unit 110 is a processing device configured to comprehensively control each unit of the terminal 100 according to various programs such as system programs stored in the storage unit 190 and perform various types of processing related to image processing, and includes a processor such as a CPU or DSP or an integrated circuit such as an ASIC.

The processing unit 110 includes, for example, a first region detection unit 111, a second region detection unit 112, a first color information calculation unit 113, a second color information calculation unit 114, a color difference calculation unit 115, and a display control unit 116. The units from the first region detection unit 111 to the second color information calculation unit 114 correspond to the above-described units from the first region detection unit 11 to the second color information calculation unit 14.

The color difference calculation unit 115 is a type of the above-described color relative relationship information calculation unit 15, and calculates a color difference between the first color information and the second color information.

The display control unit 116 controls the display unit 130 to display information on a color difference calculated by the color difference calculation unit 115 and output in time series.

For example, processing in which the processing unit 110 transmits color relative relationship information (in this example, a color difference) to each functional unit in order to perform various types of control (display control, sound output control, communication control, etc.) may be regarded as output, and the output unit 16 of the image processing apparatus 1 in FIG. 1 may be regarded as the processing unit 110 including the display control unit 116.

Also, for example, the output unit 16 of the image processing apparatus 1 in FIG. 1 may be regarded as functional units such as the display unit 130, the sound output unit 140, or the communication unit 180.

The operation unit 120 includes input devices such as operation buttons and operation switches with which the user makes various operational inputs to the terminal 100. The operation unit 120 includes the touch panel 125 integrally configured with the display unit 130, and the touch panel 125 functions as an input interface between the user and the terminal 100. The operation unit 120 outputs operation signals in accordance with user operations to the processing unit.

The display unit 130 is a display device including an LCD (Liquid Crystal Display) or the like, and displays various indications based on display signals output from the display control unit 116. In this example, the display unit 130 is integrally configured with the touch panel 125 to form a touch screen.

The sound output unit 140 is a sound output device including a speaker or the like, and outputs various sounds based on sound output signals output from the processing unit 110.

The image capturing unit 150 is an image capturing device configured to capture an image of any scene, and includes an image sensor (semiconductor device) such as a CCD (Charge Coupled Device) image sensor or a CMOS (Complementary MOS) image sensor. The image capturing unit 150 causes light emitted from an image capture target to form an image on the light-receiving plane of the image sensor by means of an unshown lens, and converts the light intensity of the image into electrical signals through photoelectric conversion. The converted electrical signals are converted to digital signals by an unshown A/D (Analog Digital) converter and output to the processing unit. The image capturing unit 150 is located, for example, on the side of the terminal 100 where the touch panel 125 is present. It may be referred to as a front camera.

The image capturing unit 150 may be configured as an image capturing unit (rear camera) that is located on the rear face of the terminal 100 where the touch panel 125 is not present and that includes a flash (strobe) configured to be used as a light source when capturing an image. This flash may be a flash whose color temperature during light emission is known or whose color temperature can be dimmed by the processing unit.

The terminal may include two image capturing units consisting of the above-described front and rear cameras.

When these image capturing units capture an image, the display control unit 116 may display the live view images on the display unit 130.

The environmental information detection unit 160 detects information on the environment of the terminal (hereinafter referred to as “environmental information”). The environmental information can include, for example, at least one of temperature and humidity.

The clock unit 170 is a built-in clock of the terminal 100 and outputs time information (time record information). The clock unit 170 includes, for example, a clock using a crystal oscillator or the like.

The clock unit 170 may include a clock to which the NITZ (Network Identity and Time Zone) standard or the like is applied.

The communication unit 180 is a communication device for transmitting and receiving information used inside the terminal to and from an external information processing apparatus. As the communication method of the communication unit 180, various methods can be used including a wired connection via a cable conforming to a predetermined communication standard, a connection via an intermediate device called a cradle that also serves as a charger, and a wireless connection using wireless communication.

The storage unit 190 is a storage device including a volatile or nonvolatile memory such as ROM, EEPROM, flash memory, or RAM, a hard disk device, or the like.

In this example, the storage unit 190 stores, for example, a color information processing program 191, a color difference calculation processing program 193, an image buffer 195, and color difference history data 197.

The color information processing program 191 is a program that is read by the processing unit 110 and executed as color information processing.

The color difference calculation processing program 193 is a program that is read by the processing unit 110 and executed as color difference calculation processing.

The image buffer 195 is, for example, a buffer in which captured images captured by the image capturing unit 150 are stored.

The color difference history data 197 is, for example, data in which the color difference calculated by the color difference calculation unit 115 is stored in association with the date and time (or time) recorded by the clock unit 170.

In addition to these functional units, for example, a gyro sensor that detects angular velocity around three axes and the like may also be provided.

### Processing

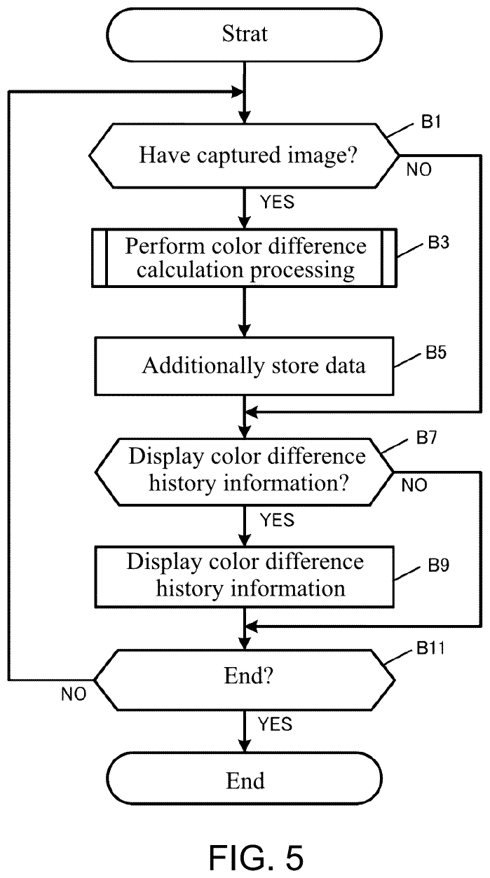

FIG. 5 is a flowchart showing an example of the flow of color information processing that is performed by the processing unit 110 of the terminal 100A in this example.

First, the processing unit 110 determines whether or not an image has been captured by the image capturing unit 150 (B1), and if it is determined that an image has been captured (B1: YES), the processing unit stores data of the captured image in the image buffer 195. Then, the color difference calculation unit 115 performs color difference calculation processing (B3). Specifically, a color difference is calculated as the color relative relationship information from the above-mentioned captured image based on the processing given as an example in FIG. 3 or the like. The processing unit 110 stores the calculated color difference in the color difference history data 197 in association with the time record information (date and time, etc.) of the clock unit 170 (B5).

The processing unit 110 may acquire data of a plurality of captured images from the image capturing unit 150, and calculate color relative relationship information (a color difference) based on the data of the plurality of captured images stored in the image buffer 195.

Subsequently, the processing unit 110 determines whether or not to display color difference history information (B7). Specifically, for example, it is determined whether or not an input has been made by the user to display color difference history information, via the operation unit 120, the touch panel 125, or the like.

If it is determined that color difference history information is to be displayed (B7: YES), the processing unit displays color difference history information on the display unit 130 based on a color difference history stored in the color difference history data 197 (B9).

Next, the processing unit 110 determines whether or not to end the processing, and if it is determined that the processing is to be continued (B11: NO), the processing returns to B1.

On the other hand, if it is determined that the processing is to be ended (B11: YES), the processing unit 110 ends the processing.

If it is determined that an image has not been captured by the image capturing unit 150 (B1: NO), the processing unit 110 advances the processing to B7.

If it is determined that color difference history information is not to be displayed (B7: NO), the processing unit 110 advances the processing to B11.

When displaying the color difference history information, the terminal 100A may display the color difference in association with the date and time or the time at which the image was captured by the image capturing unit 150, based on the time record information of the clock unit 170.

Furthermore, when displaying the color difference history information, the terminal 100A may display the color difference and the environmental information detected by the environmental information detection unit 160 in association with each other. More specifically, the color difference and the environmental information may be displayed so as to correspond to the date and time or the time, using the detection result of the environmental information detection unit 160 at the date and time or the time corresponding to the date and time or the time at which the image was captured by the image capturing unit 150.

It is also possible to display only one of temperature and humidity as the environmental information. The environmental information may be acquired from an unshown environmental information provision server via the communication unit 180.

In the above processing, an example was shown in which a color difference (a scalar value) is calculated and displayed as the color relative relationship information, but there is no limitation to this. As described above, a color relative relationship vector may be calculated and displayed as the color relative relationship information. For example, the color relative relationship vector may be displayed as an arrow in the color space.

These display screen examples will be illustrated and explained as display screens of the skin analysis application in the next example.

### (2) Example with Terminal and Server

Next, an example of a server to which the above-described image processing apparatus 1 is applied or that includes the above-described image processing apparatus 1, and a system including the server and a terminal will be described. The server can be, for example, a server (e.g., a server in a client-server system) that communicates with the user's terminal described above.

Hereinafter, an example in which the user's terminal communicates with the server and displays color relative relationship information using an application that analyzes human skin (hereinafter referred to as a “skin analysis application”) will be described as an example of a client-server system.

The skin analysis application (application program) may be an application that is downloaded from a server, stored in the storage unit of the terminal, and then executed, or may be an application that is executed without the need to download (e.g., a Web application).

### Functional Configuration

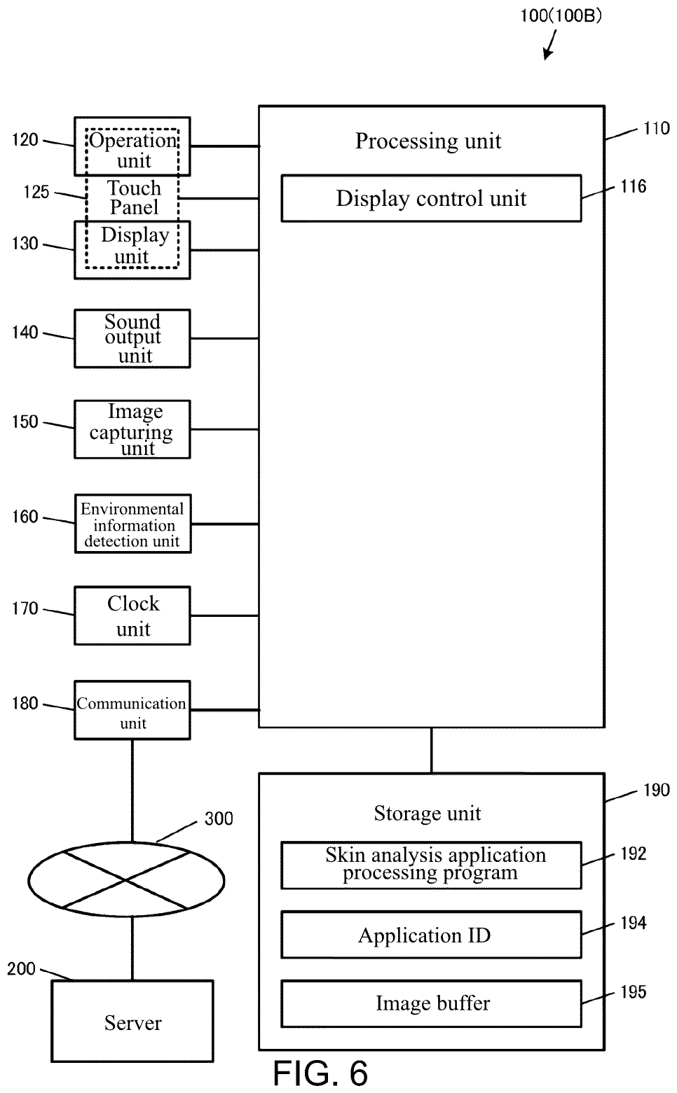

FIG. 6 is a diagram showing an example of the functional configuration of a terminal 100B, which is an example of the terminal 100 in this example.

The constituent elements that are the same as those of the terminal 100A shown in FIG. 4 are denoted by the same reference numerals, and redundant descriptions thereof have been omitted.

In this example, the processing unit 110 of the terminal 100B includes, for example, the above-described display control unit 116 as a functional unit.

In this example, the communication unit 180 of the terminal 100B communicates with a server 200 configured to manage various types of information on the skin analysis application, via a network 300.

In this example, the storage unit 190 of the terminal 100B stores, for example, a skin analysis application processing program 192 that is read by the processing unit 110 and executed as skin analysis application processing, an application ID 194 that is information on the account of the terminal 100B that uses the skin analysis application or the user of the terminal 100B, and the above-described image buffer 195.

In this example, the terminal 100B may not include the environmental information detection unit 160, and may acquire environmental information, for example, from the server 200.

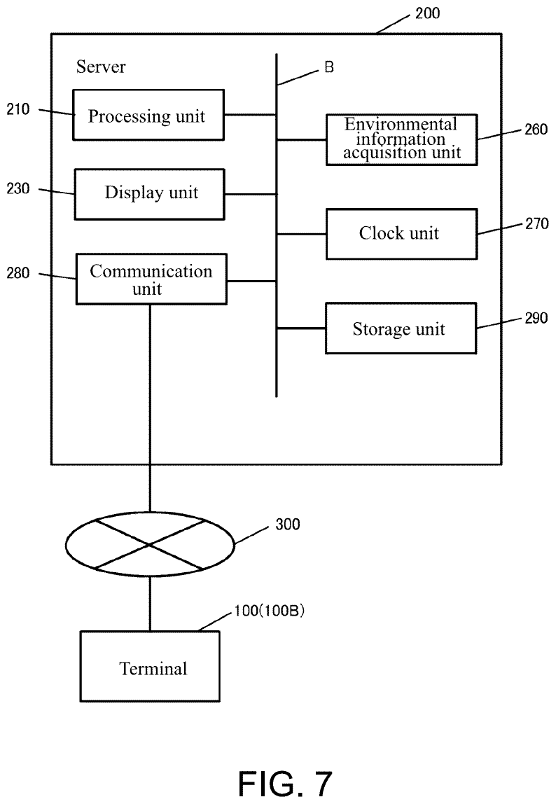

FIG. 7 is a diagram showing an example of the functional configuration of the server 200 in this example.

The server 200 includes, for example, a processing unit 210, a display unit 230, an environmental information acquisition unit 260, a clock unit 270, a communication unit 280, and a storage unit 290, and these units are connected to each other via a bus B.

The HW configuration of the processing unit 210, the display unit 230, the clock unit 270, the communication unit 280, and the storage unit 290 can be the same as that of the terminal 100, and thus descriptions thereof have been omitted.

The environmental information acquisition unit 260 acquires environmental information, for example, detected by an environmental information detection unit (a temperature sensor, a humidity sensor, etc.) included in the server, or acquires environmental information from an unshown environmental information provision server configured to provide environmental information. If environmental information is acquired from the environmental information provision server, the communication unit may be regarded as the environmental information acquisition unit.

The communication unit 280 transmits and receives information (data) to and from other devices including the terminal 100B, via the network 300 under the control of the processing unit 210.

The storage unit 290 stores, for example, management data related to the terminal 100B that uses the skin analysis application or the user of the terminal 100B.

The storage unit 290 stores, for example, color difference history data in the form of a database in which a color difference is stored in association with time record information of the clock unit 270 for each application ID.

In this example, the server 200 may not include the environmental information acquisition unit 260, and may acquire environmental information from the terminal 100B.

### Processing

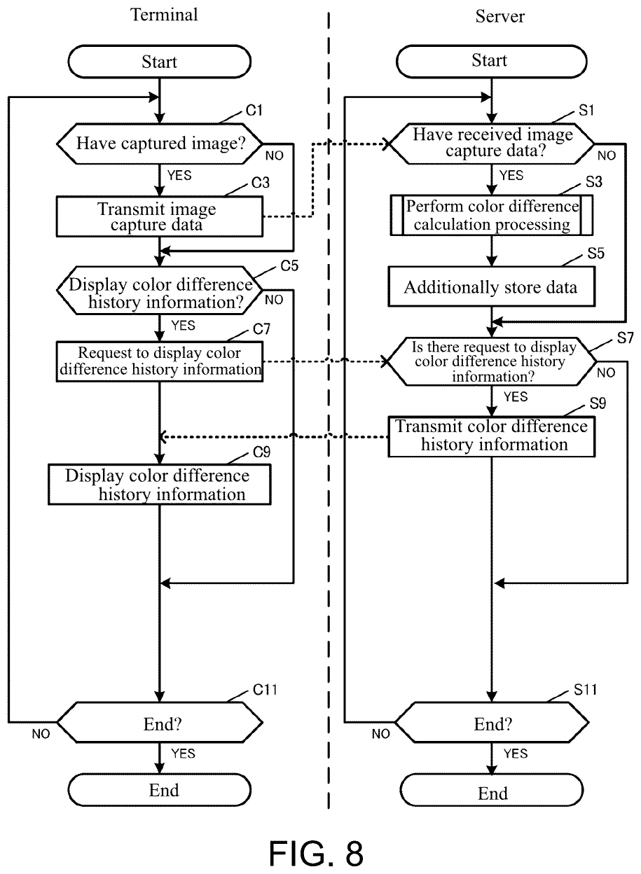

FIG. 8 is a flowchart showing an example of the flow of processing that is performed by each device in this example. In this diagram, the left portion shows an example of skin analysis application processing that is performed by the processing unit 110 of the terminal 100B, and the right portion shows an example of management processing related to a skin analysis application that is executed by the processing unit 210 of the server 200.

First, the processing unit 110 of the terminal 100B determines whether or not an image has been captured by the image capturing unit 150 through the skin analysis application (C1). If it is determined that an image has been captured (C1: YES), the processing unit 110 of the terminal 100B causes, for example, the communication unit 180 to transmit image capture data containing the application ID 194 stored in the storage unit 190 and data of the captured image, to the server 200 (C3). The processing unit 110 of the terminal 100B may repeatedly cause the image capturing unit 150 to capture an image, and transmit image capture data containing data of a plurality of captured images.

The processing unit 210 of the server 200 determines whether or not image capture data from the terminal 100B has been received by the communication unit 280 (S1), and if it is determined that image capture data has been received (S1: YES), the processing unit performs, for example, processing for calculating a color difference between the first color information and the second color information from the captured image contained in the image capture data received from the terminal 100B, according to an unshown color difference calculation processing program stored in the storage unit 290 (S3). Then, the processing unit 210 of the server 200 stores the calculated color difference as color difference history data corresponding to the application ID contained in the received image capture data, in association with the time record information (date and time, etc.) of the clock unit 270 (S5).

After C3, the processing unit 110 of the terminal 100B determines whether or not to display color difference history information through the skin analysis application (C5). Specifically, for example, it is determined whether or not an input has been made by the user to display color difference history information, via the operation unit 120, the touch panel 125, or the like, through the skin analysis application.

If it is determined that color difference history information is to be displayed (C5: YES), the processing unit 110 of the terminal 100B causes, for example, the communication unit 180 to transmit a color difference history information display request containing the application ID 194 stored in the storage unit 190 and information requesting display of color difference history information, to the server 200 (C7).

After S5, the processing unit 210 of the server 200 determines whether or not a color difference history information display request has been received from the terminal 100B (S7), and if it is determined that the request has been received (S7: YES), the processing unit causes, for example, the communication unit 280 to transmit color difference history information containing a set period of a color difference history based on the color difference history data stored in the storage unit 290 and corresponding to the received application ID 194, to the terminal 100B (S9).

After C7, if the communication unit 180 receives the color difference history information from the server 200, the processing unit 110 of the terminal 100B causes the display unit 130 to display the received color difference history information through the skin analysis application (C9).

Subsequently, the processing unit 110 of the terminal 100B determines whether or not to end the processing (C11), and if it is determined that the processing is to be continued (C11: NO), the processing returns to C1.

On the other hand, if it is determined that the processing is to be ended (C11: YES), the processing unit 110 of the terminal 100B ends the skin analysis application processing.

After S9, the processing unit 210 of the server 200 determines whether or not to end the processing (S11), and if it is determined that the processing is to be continued (S11: NO), the processing returns to S1.

On the other hand, if it is determined that the processing is to be ended (S11: YES), the processing unit 210 of the server 200 ends the processing.

If it is determined that an image has not been captured by the image capturing unit 150 (C1: NO), the processing unit 110 of the terminal 100B advances the processing to C5.

If it is determined that color difference history information is not to be displayed (C5: NO), the processing unit 110 of the terminal 100B advances the processing to C11.

If it is determined that image capture data has not been received (S1: NO), the processing unit 210 of the server 200 advances the processing to S7.

If it is determined that a color difference history information display request has not been received (S7: NO), the processing unit 210 of the server 200 advances the processing to S11.

In this processing, only one terminal 100B is shown, but the terminals 100 using the skin analysis application can perform similar processing. The server 200 can also perform similar processing for each of the terminals 100.

In the above processing, an example was shown in which a color difference (a scalar value) is calculated and displayed as the color relative relationship information, but there is no limitation to this. As described above, a color relative relationship vector may be calculated and displayed as the color relative relationship information. For example, the color relative relationship vector may be displayed as an arrow in the color space.

### Display Screen

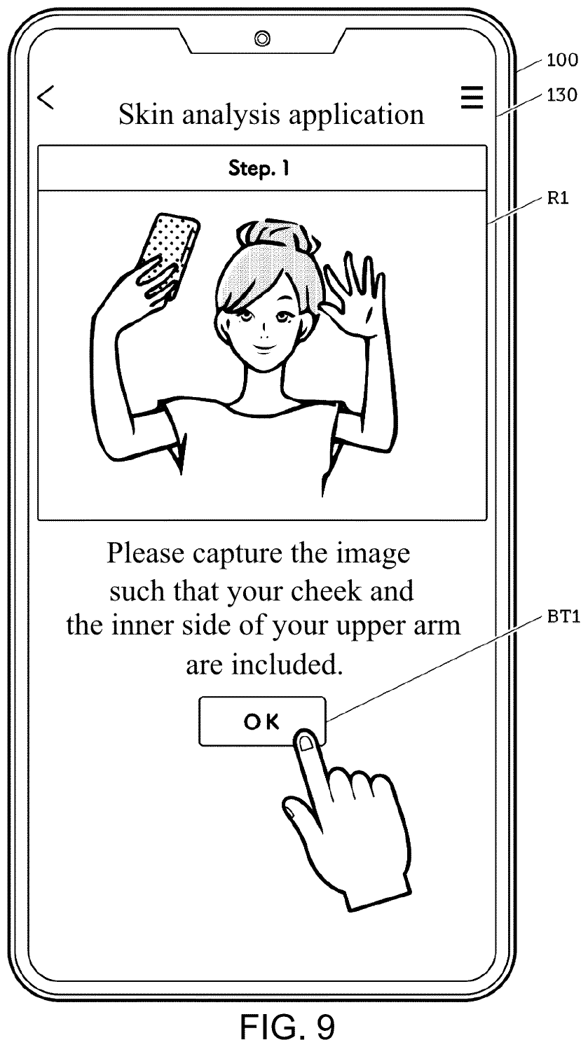

FIG. 9 is a diagram showing an example of a screen of the skin analysis application displayed on the display unit 130 of the terminal 100 in this example.

This screen shows an example of a navigation screen that is displayed when an input has been made by the user to start the image capturing unit 150 in the skin analysis application (B1 in FIGS. 5 and C1 in FIG. 8), and the upper portion in the screen shows a navigation region R1 to provide an illustration of how to capture an image, along with the text “Step. 1”. This illustration shows a woman capturing an image while holding the terminal 100 with her right hand and raising the opposite arm. The text “Please capture the image such that your cheek and the inner side of your upper arm are included” is displayed for more detailed navigation below the illustration, and an “OK” button BT1 for proceeding to the next screen is displayed below the text.

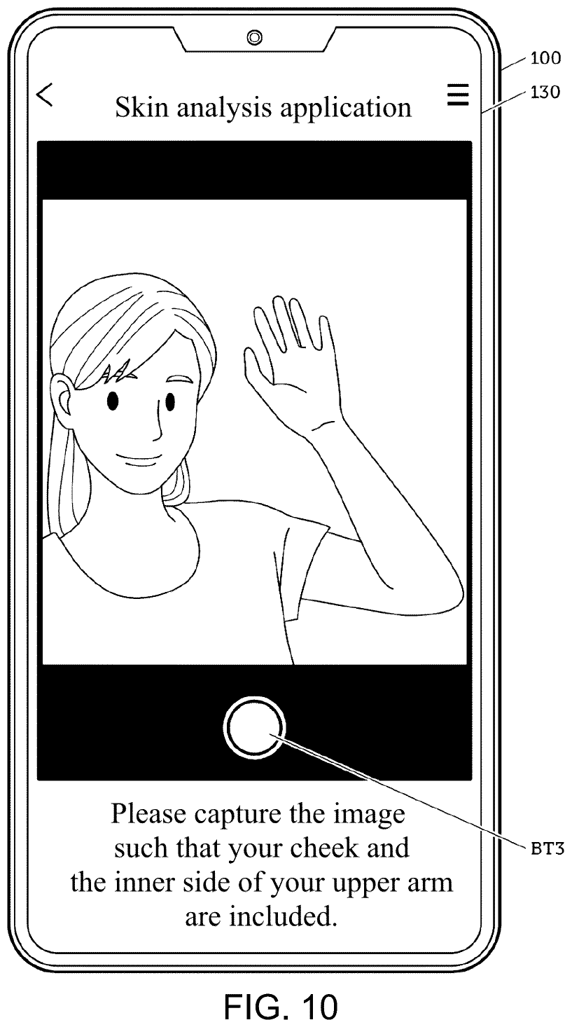

When the “OK” button BT1 is tapped, the display switches to the screen shown in FIG. 10, for example (B1 in FIGS. 5 and C1 in FIG. 8).

This screen shows an example of an image capture screen for the user to capture an image in the skin analysis application, in which, for example, a live view image of the front camera and an image capture button BT3 shown, for example as a concentric circle for capturing an image are arranged. The text “Please capture the image such that your cheek and the inner side of your upper arm are included” is displayed below the button as in FIG. 9. On this screen, the user can tap the image capture button BT3 to cause the image capturing unit 150 to capture an image while making a pose shown in FIG. 9.

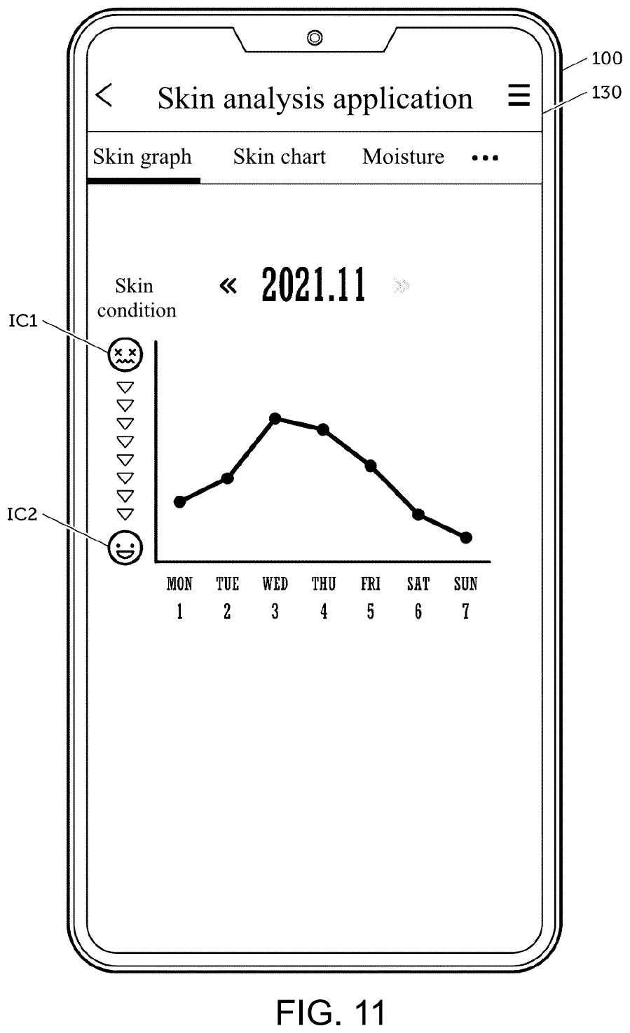

FIG. 11 is a diagram showing an example of a color difference history information display screen that is displayed when an input has been made by the user to display color difference history information in the skin analysis application (B9 in FIGS. 5 and C5: YES to C9 in FIG. 8).

This screen shows a state in which the tab “skin graph” has been tapped among the tabs with a plurality of functions that are displayed in the upper portion in the screen and available to the user in the skin analysis application.

The center portion in the screen displays a graph of a color difference history over the past predetermined period (in this example, the period from “Nov. 1, 2021” to “Nov. 7, 2021”). In this example, the user captured an image every day during the above 7-day period, and the daily color difference transition is displayed in the form of a graph (in this example, a line graph) in which the horizontal axis indicates the date and the vertical axis indicates the color difference.

The graph is not limited to line graphs, and may be displayed as bar graphs or other forms of graphs. Numerical values may also be displayed instead of graphs.

Furthermore, in this example, the text “skin condition” is displayed in association with the vertical axis. A “bad” icon IC1 (in this example, an icon of a face that is not smiling) indicating that the skin condition is bad is displayed next to the top of the vertical axis, and a “good” icon IC2 (in this example, an icon of a face that is smiling) indicating that the skin condition is good is displayed next to the bottom of the vertical axis. Also, a plurality of inverted triangular marks indicating that the skin condition is getting better step by step are displayed from the “bad” icon IC1 to the “good” icon IC2.

In this example, a user's cheek region is taken as a determination target region (measurement target region). Also, the inner region of a user's upper arm is taken as a comparison target region. Then, the above-described processing is performed in which the user's cheek region is taken as the first region and the user's upper arm region is taken as the second region.

As described above, the inner side of an upper arm is considered to be less prone to external disturbances such as sunlight and to be likely to maintain its skin color. Therefore, in this example, the color of the inner side of an upper arm is set as the user's target skin color (ideal value). That is to say, the color of the inner region of an upper arm is set as the benchmark for whitening. The user is notified of the color difference as an index value to indicate how much the color of the user's cheek deviates from the color of the inner side of the upper arm, based on the captured images captured by the user.

According to the description above, a smaller color difference can be considered to indicate that the color of the user's cheek is closer to the ideal value. Conversely, a larger color difference can be considered to indicate that the color of the user's cheek is farther from the ideal value.

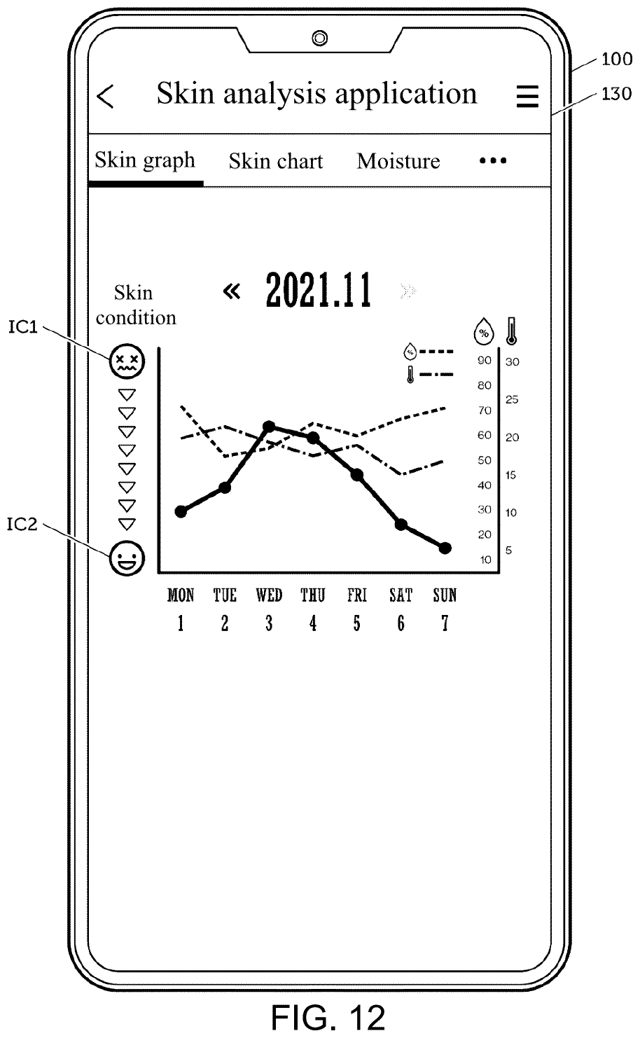

FIG. 12 is a diagram showing another example of the color difference history information display screen in FIG. 11.

This screen shows time-series graphs of temperature and humidity as environmental information in association with the time-series graph of color difference shown in FIG. 11. That is to say, the color difference information and the environmental information are displayed on the same timeline.

Such a display allows the user to compare the time-series color difference with the time-series temperature and humidity, and enables the user to analyze the type of environment in which the skin condition tends to be good and the type of environment in which the skin condition tends to be bad.

It is also possible to display only one of temperature and humidity as the environmental information.

In this case, for example, in Step S9 in FIG. 8, the processing unit 210 of the server 200 can transmit the environmental information acquired by the environmental information acquisition unit 260 to the terminal 100, along with the time-series color difference information. In this case, the server 200 may acquire environmental information in the area in which the user resides and at the location of the terminal 100 based on information on the area registered in advance and information on the location of the terminal 100 transmitted from the terminal 100, and transmit the information to the terminal 100. That is to say, in this example, the terminal 100 can acquire environmental information from the server 200.

Instead of this configuration, the server 200 may receive image capture data containing environmental information from the terminal 100 (C3 and S1: YES in FIG. 8), and store the environmental information received from the terminal 100, in the storage unit 290. The information in which the color difference history information is associated with the environmental information may be transmitted to the terminal 100 (S9 in FIG. 8).

### Action and Effect of Foregoing Example

In the foregoing example, the terminal (an example of the image processing apparatus) calculates first color information in a first region included in a captured image and second color information in a second region included in the captured image. Then, the terminal calculates color relative relationship information (an example of relative relationship information on a relative relationship) between the calculated first color information and second color information. Then, the terminal outputs time-series color differences for a plurality of captured images captured at different points in time.

Accordingly, the terminal can calculate the relative relationship information on a relative relationship between the first color information in the first region included in the captured image and the second color information in the second region included in the captured image. It is possible to output the time-series relative relationship information for a plurality of captured images captured at different points in time.

Furthermore, in this case, the color relative relationship information may be, for example, color difference information.

Accordingly, the terminal can output the time-series color difference information for a plurality of captured images captured at different points in time.

Furthermore, in this case, the first region and the second region may be different regions in the same subject.

Accordingly, it is possible to calculate color relative relationship information based on color information in different regions in the same subject. Also, for example, the second region can be set as a comparison target region and used as a guide to determine how much the color of the first region in the same subject deviates from the color of the second region in the same subject.

Furthermore, in this case, the first region may be a face region.

Accordingly, it is possible to obtain a guide to determine how much the color of the face region in a particular subject deviates from the color of a region that is in the same subject and different from the face region.

Furthermore, in this case, the first region may be a cheek region.

Accordingly, it is possible to obtain a guide to determine how much the color of the cheek region in a particular subject deviates from the color of a region that is in the same subject and different from the face region.

Furthermore, in this case, the first region may be a neck region.

Accordingly, it is possible to obtain a guide to determine how much the color of the neck region in a particular subject deviates from the color of a region that is in the same subject and different from the neck region.

Furthermore, in this case, the second region may be an inner region of an arm.

Accordingly, it is possible to obtain a guide to determine how much the color of a region that is in a particular subject and different from the inner side of an arm deviates from the color of the inner region of the arm in the same subject.

Furthermore, in this case, the second region may be an inner region of an upper arm or an inner region of a wrist.

Accordingly, it is possible to obtain a guide to determine how much the color of a region that is in a particular subject and different from the inner region of an upper arm deviates from the color of the inner region of the arm in the same subject. It is possible to obtain a guide to determine how much the color of a region that is in a particular subject and different from the inner region of a wrist deviates from the color of the inner region of the wrist in the same subject. Furthermore, in the foregoing example, the user's terminal may include any one of the above-described image processing apparatuses, the image capturing unit 150 configured to capture an image, and the display unit 130 configured to display color relative relationship information. Accordingly, it is possible for the user to recognize relative relationship information calculated based on a captured image captured by the image capturing unit and displayed on the display unit.

Furthermore, in this case, the display unit 130 may display the color relative relationship information in association with information on the date and time or the time at which the image was captured (an example of information for specifying a point in time).

Accordingly, it is possible for the user to recognize the relative relationship information along with the point in time at which the image was captured. Since the user can refer to the point in time at which the image was captured, the convenience for the user can be improved.

Furthermore, in this case, the user's terminal may further include the communication unit 180 (an example of the environmental information acquisition unit configured to acquire environmental information) configured to receive environmental information from the environmental information detection unit 160 or a server, and the display unit 130 may display color relative relationship information in association with the environmental information.

Accordingly, it is possible for the user to recognize the relative relationship information along with the acquired environmental information. Since the user can refer to the environmental information, the convenience for the user can be improved.

Furthermore, in the foregoing example, the server (an example of the image processing apparatus) calculates first color information in a first region included in a captured image and second color information in a second region included in the captured image. Then, the server calculates color relative relationship information (an example of relative relationship information on a relative relationship) between the calculated first color information and second color information. Then, the server outputs time-series color differences for a plurality of captured images captured at different points in time.

Accordingly, the server can calculate the relative relationship information on a relative relationship between first color information in a first region included in a captured image and second color information in a second region included in the captured image. It is possible to output the time-series relative relationship information for a plurality of captured images captured at different points in time.

Furthermore, in the foregoing example, the server includes any one of the above-described image processing apparatuses, and a communication unit configured to receive a captured image from a terminal and transmit calculated relative relationship information to the terminal.

Accordingly, the server can acquire a captured image from a terminal, and calculate relative relationship information based on the captured image and transmit the calculated information to the terminal. From the terminal's perspective, the terminal only needs to transmit captured images to the server and does not need to perform the calculation, and thus it is possible to reduce the processing load.

### Other Examples

Hereinafter, other examples (modified examples) will be described.

### (1) Mode

The foregoing example may be applied to, merely in an example, a case in which an image is captured when the user is in a no-makeup state (without wearing makeup).

However, there is no limitation to this, and, for example, the foregoing example may be applied to a case in which an image is captured when foundation has been applied on the user, for example.

For example, a mode in which an image is captured in the no-makeup state is referred to as a “no-makeup mode”, and a mode in which an image is captured with foundation applied is referred to as a “foundation mode”. For example, the user may select the “no-makeup mode” or the “foundation mode” for capturing an image.

In this case, although not shown, for example, a user interface (UI) is configured that allows the user to select the “no-makeup mode” or the “foundation mode” before the screen shown in FIG. 9 is displayed or within the screen shown in FIG. 9. If the “no-makeup mode” is selected, the user captures an image in a no-makeup state, on the image capture screen shown in FIG. 10. If the “foundation mode” is selected, the user captures an image with foundation applied, on the image capture screen shown in FIG. 10.

Then, for each mode, processing similar to that described above can be performed on a captured image captured in that mode and the result can be displayed on the terminal 100.

Specific examples thereof will be described in conjunction with the following example.

### (2) Notification to User

The terminal 100 may make a notification to the user based on the color relative relationship information.

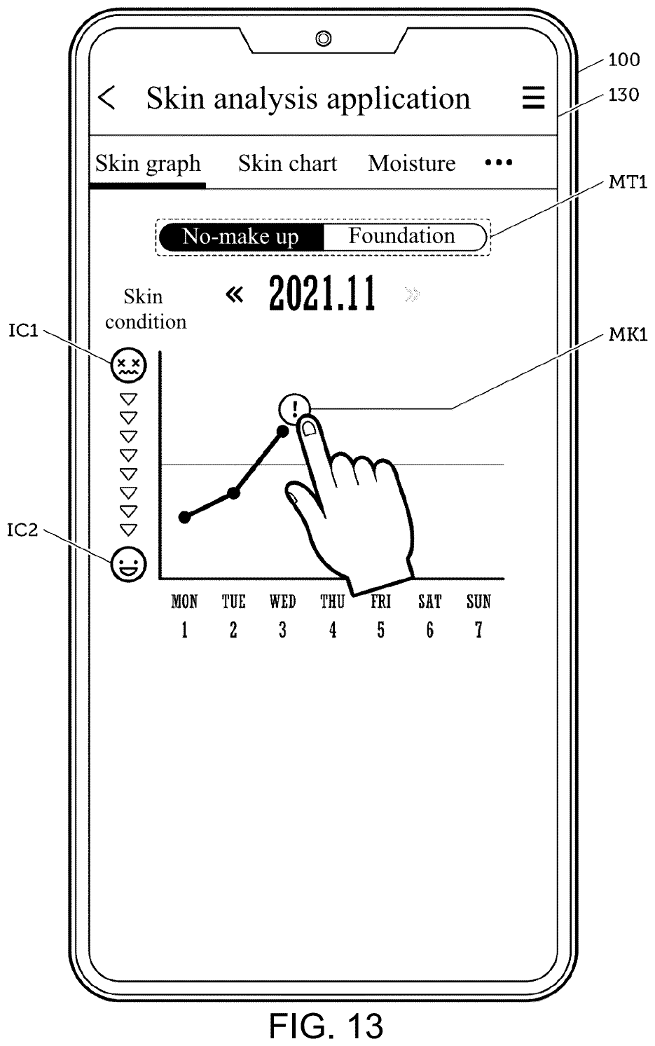

FIG. 13 is a diagram showing an example of a color difference history information display screen displayed on the display unit 130 of the terminal 100 in this example. The details of the screen are the same as those in FIG. 11.

As one example, a case will be described in which notifications with different notification contents are respectively made for the two modes “no-makeup mode” and “foundation mode” described in “(1) Mode” above.

This screen in FIG. 13 shows a mode switching tab MT1 including a tab for displaying color difference history information based on captured images captured in the “no-makeup mode” and a tab for displaying color difference history information based on captured images captured in the “foundation mode”, below the region including the tabs with a plurality of functions that are displayed in the upper portion in the screen and available to the user in the skin analysis application. In this example, the tab “no-makeup mode” is highlighted and the color difference history information calculated in the “no-makeup mode” is displayed below this tab, in response to the user tapping the tab “no-makeup mode” in the mode switching tab MT1.

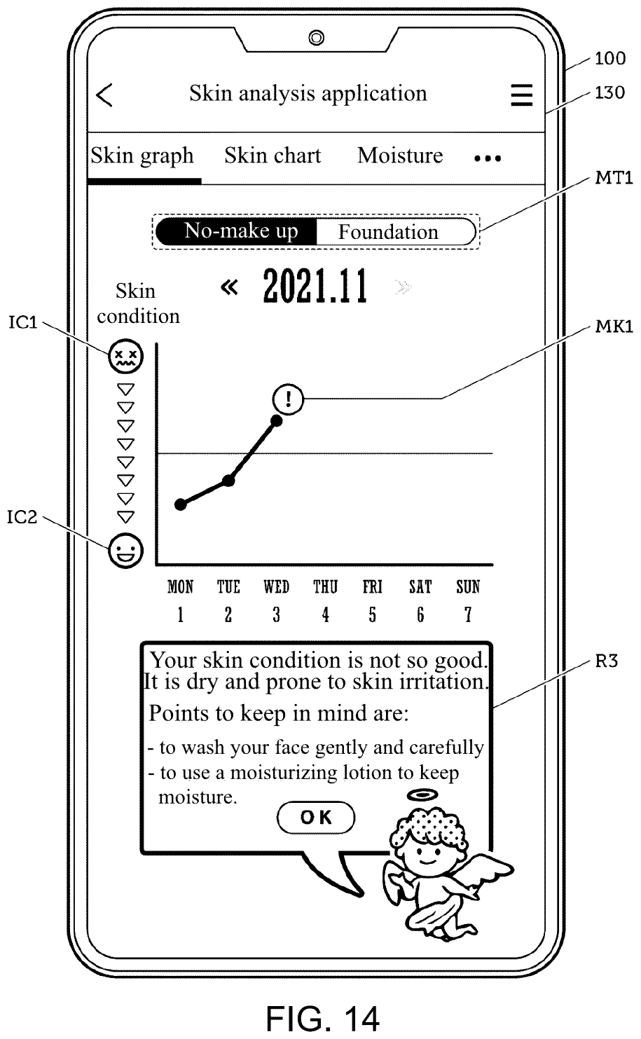

This example shows a case in which the color difference is greater than or equal to a set value (threshold value) or is greater than the set value in the graph of time-series color differences. In this example, a state is shown in which the color difference calculated based on a captured image captured on “Nov. 3, 2021” is greater than or equal to a set value or is greater than the set value. In this example, the caution mark MK1 is displayed next to the color difference value. When this caution mark MK1 is tapped by the user, for example, the screen as shown in FIG. 14 is displayed.

In this screen, the region for displaying notification information transmitted from the server is configured below the region displaying the graph, in response to the caution mark MK1 being tapped. In this example, a region R3 represented by the balloon from the angel illustration in the lower portion in the screen displays information containing the text “Your skin condition is not so good. It is dry and prone to skin irritation.”, the text that tells the user about the points to keep in mind, and an “OK” button to hide these displays.

That is to say, in this example, notification information to the user is displayed on the display unit 130 in response to a set condition that the color difference is greater than or equal to a first set value or is greater than the first set value being satisfied.

Specifically, as the notification information, information to alert the user to the fact that the first color information deviates from an ideal that is the second color information, more specifically, information to alert the user to the skin condition (information to notify the user of the fact that the skin condition is not good) is displayed.

Although not shown, contrary to the above-described example, notification information to the user may be displayed on the display unit 130 in response to a set condition that the color difference is less than a second set value or is less than or equal to the second set value being satisfied. The second set value may be set as a value that is smaller than the first set value.

Specifically, for example, as the notification information, information to inform the user of the fact that the first color information is close to an ideal that is the second color information, more specifically, information to inform the user of the fact that the skin condition is good may be displayed.

Furthermore, the above-described display pattern of notification is merely an example, and there is no limitation to this.

For example, in response to the caution mark MK1 being tapped, notification information may be displayed in the form of a balloon, a speech bubble, or the like based on the caution mark MK1 or the color difference value to which the caution mark MK1 is attached.

The notification information may be displayed without displaying the caution mark MK1.

Furthermore, the above-described notification is not limited to being made by displaying, but may also be realized by causing the sound output unit 140 to output a sound (including speech). That is to say, the notification information may be sound information (including speech information).

For example, a caution sound or an alert announcement may be output from the sound output unit 140 in response to a set condition that the color difference is greater than or equal to a first set value or is greater than the first set value being satisfied. The alert announcement may have the content that is the same as or different from that shown in text in FIG. 14.

Also, for example, a fanfare sound or a blessing announcement may be output from the sound output unit 140 in response to a set condition that the color difference is less than a second set value or is less than or equal to the second set value being satisfied.

Furthermore, the mode switching tab MT1 does not have to be displayed on the screen in FIGS. 13 and 14 if the application is not provided with the “no-makeup mode” and the “foundation mode” as described above and is basically designed to capture an image in a no-makeup state.

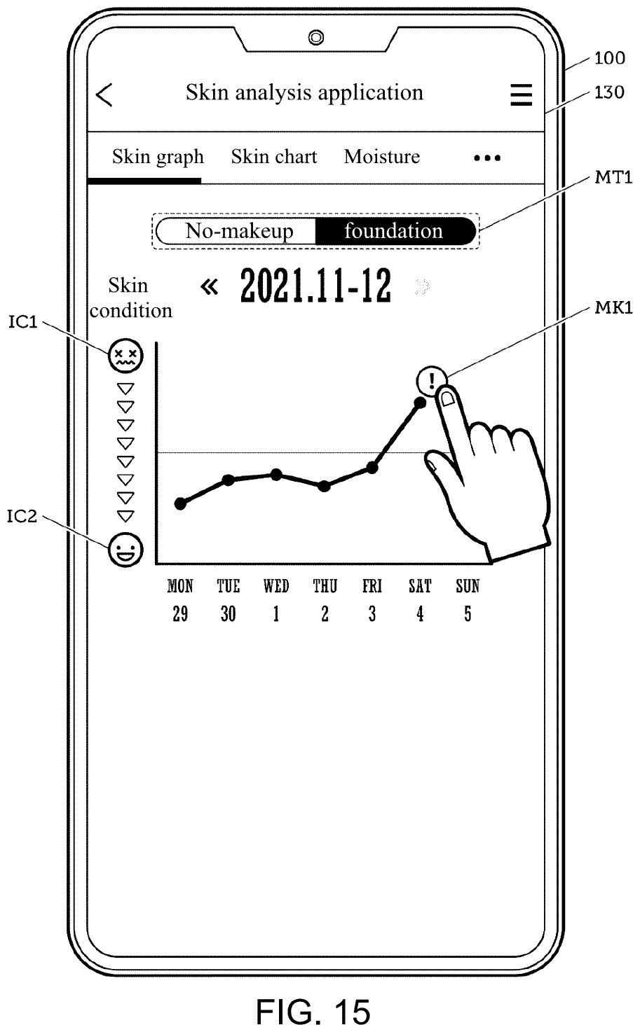

FIG. 15 is a diagram showing an example of a color difference history information display screen displayed on the display unit 130 of the terminal 100 in this example.

In this screen, the tab of the foundation mode is highlighted and the color difference history information in the foundation mode is displayed below this tab, in response to the user tapping the tab of the foundation mode in the mode switching tab MT1.

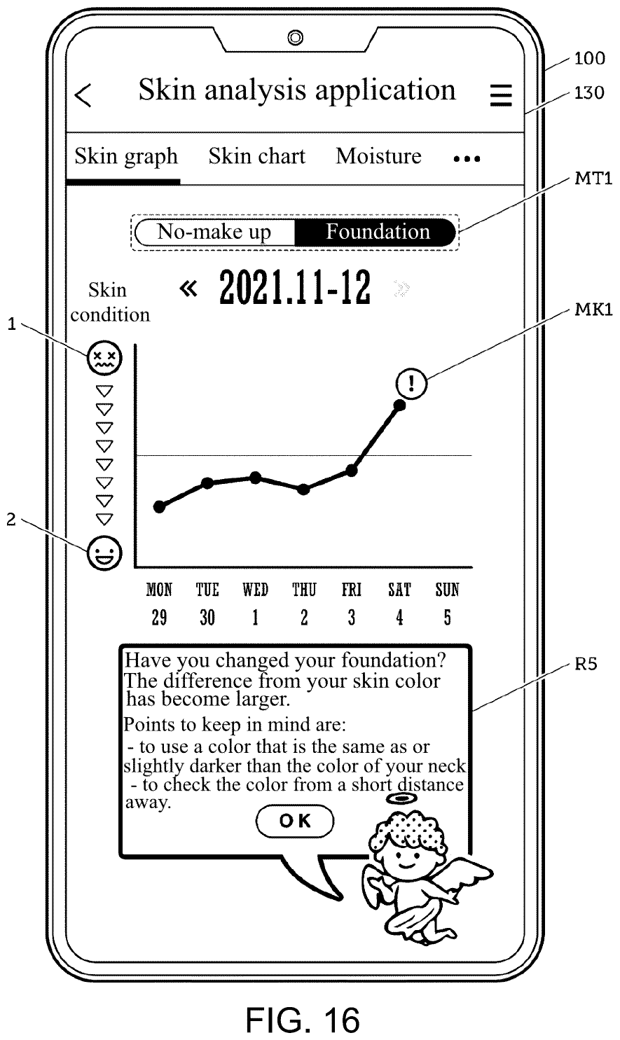

This example shows as with FIG. 13 a case in which the color difference is greater than or equal to a set value or is greater than the set value in the graph of time-series color differences in the foundation mode. In this example, a state is shown in which the color difference calculated based on a captured image captured on “Dec. 4, 2021” in the foundation mode is greater than or equal to a set value or is greater than the set value. In this example, the above-mentioned caution mark MK1 is displayed next to the color difference. When this caution mark MK1 is tapped by the user, for example, the screen as shown in FIG. 16 is displayed.

In this screen, the region for displaying notification information transmitted from the server is configured below the region displaying the graph. In this example, a region R5 represented by the balloon from the angel illustration in the lower portion in the screen as with FIG. 14 displays information containing the text “Have you changed your foundation? The difference from your skin color has become larger.”, the text that tells the user about the points to keep in mind, and an “OK” button to hide these displays.

Since this is the “foundation mode”, the notification content is different from that in the “no-makeup mode” in FIG. 14, and notification information corresponding to the “foundation mode” is displayed.

That is to say, in this example, notification information to the user is displayed on the display unit 130 in response to a set condition that the color difference calculated in the “foundation mode” is greater than or equal to a third set value or is greater than the third set value being satisfied.

Specifically, as the notification information, information to alert the user to the fact that the first color information deviates from an ideal that is the second color information, more specifically, for example, information to alert the user to the fact that the foundation (the color of the foundation) does not suit the user or the like is displayed.

Although not shown, contrary to the above-described example, notification information to the user may be displayed on the display unit 130 in response to a set condition that the color difference calculated in the “foundation mode” is less than a fourth set value or is less than or equal to the fourth set value being satisfied. The fourth set value may be set as a value that is smaller than the third set value.

Specifically, for example, as the notification information, information to inform the user of the fact that the first color information is close to an ideal that is the second color information, more specifically, for example, information to inform the user of the fact that the foundation (the color of the foundation) suits the user may be displayed.

Furthermore, the above-described display pattern of notification is merely an example, and there is no limitation to this.

For example, in response to the caution mark MK1 being tapped, notification information may be displayed in the form of a balloon, a speech bubble, or the like based on the caution mark MK1 or the color difference value to which the caution mark MK1 is attached.

The notification information may be displayed without displaying the caution mark MK1.

Furthermore, the above-described notification is not limited to being made by displaying, but may also be realized by causing the sound output unit 140 to output a sound (including speech). That is to say, the notification information may be sound information (including speech information).

For example, a caution sound or an alert announcement may be output from the sound output unit 140 in response to a set condition that the color difference is greater than or equal to a third set value or is greater than the third set value being satisfied. The alert announcement may have the content that is the same as or different from that shown in text in FIG. 16.

Also, for example, a fanfare sound or a blessing announcement may be output from the sound output unit 140 in response to a set condition that the color difference is less than a fourth set value or is less than or equal to the fourth set value being satisfied.

When the tab “no-makeup mode” in the mode switching tab MT1 shown in the above-described display screen is tapped by the user, the tab “no-makeup mode” may be highlighted and the color difference history information in the “no-makeup mode” may be displayed. In this case, for example, a screen as shown in FIG. 14 above may be displayed, and if the caution mark MK1 is included in the screen, for example, a screen as shown in FIG. 15 above may be displayed in response to the caution mark MK1 being tapped.

The content that is to be notified of (notification information) may be stored in the form of a database, for example, in the storage unit of the terminal 100 or the server 200 in association with each set value. Then, according to the set value for the threshold value condition that is satisfied by the color difference, the corresponding notification information may be read from the database and displayed.

The color difference history information in the “no-makeup mode” and the color difference history information in the “foundation mode” may be displayed in one graph (displayed such that one is overlaid on top of another) on the same timeline. Furthermore, the above-described environmental information may be displayed as well (displayed such that one is overlaid on top of another) on the same timeline.

In this example, if the color difference (an example of the relative relationship information) satisfies a set condition, the display unit 130 of the user's terminal displays notification information to the user.

Accordingly, if the relative relationship information satisfies a set condition, a notification can be made to the user by displaying the notification information.

Furthermore, in this case, the set condition may include that the color difference is greater than or equal to a first set value or is greater than the first set value, and the notification information may include information to alert the user to the fact that the first color information deviates from an ideal that is the second color information.

Accordingly, it is possible to alert the user to the fact that the first color information deviates from the ideal, when the color difference is large to some extent.

Furthermore, in this case, the notification information may include information to alert the user to the user's skin condition.

Accordingly, it is possible to alert the user to the user's skin condition, when the color difference is large to some extent.

Furthermore, in the description above, the set condition may include that the color difference is less than a second set value, which is smaller than the first set value, or is less than or equal to a second set value, which is smaller than the first set value, and the notification information may include information to inform the user of the fact that the first color information is close to an ideal that is the second color information.

Accordingly, it is possible to notify the user of the fact that the first color information is close to the ideal, when the color difference is small to some extent.

Furthermore, in this case, the notification information may include information to inform the user of the fact that the user's skin condition is good.

Accordingly, it is possible to notify the user of the fact that the user's skin condition is good, when the color difference is small to some extent.

### (3) Stepwise Notification

With regard to the above-described notification, for example, stepwise set values may be set as the set value, and a notification may be made using different notification information according to the set value that the color difference is greater than or equal to or the set value that the color difference is greater than. As described above, the notification may be made by displaying or by sound output.

For example, in the “no-makeup mode” above, if the color difference is greater than or equal to a set value A, which is set as the lowest set value, (or is greater than the set value A), the terminal displays notification information to alert the user to the fact that “the skin condition is getting a little worse”. If the color difference is greater than or equal to a set value B, which is larger than the set value A, (or is greater than the set value B), the terminal may display notification information to alert the user to the fact that “the skin condition is getting even worse”.

This is also applicable to cases where the color difference is less than a set value (or is less than or equal to the set value). In this case, with respect to the stepwise set values, the terminal may make a notification to the effect that the skin condition is getting better in accordance with a decrease in the set value that the color difference is less than (or the set value that the color difference is less than or equal to).

This content is also applicable to the “foundation mode”.

### (4) Region Setting

It may be possible, for example, to allow the user of the terminal 100 to set which region is to be used as an object region in the processing, by making a setting input to the terminal 100. For example, if the user wants to know the result when a cheek is taken as the first region and the inner side of a wrist is taken as the second region, the user may select the cheek as the first region and the inner side of the wrist as the second region and have the terminal 100 set these regions.

Also, for example, if the user wants to know the result when the neck is taken as the first region and the inner side of an upper arm is taken as the second region, the user may select the neck as the first region and the inner side of the upper arm as the second region and have the terminal 100 set these regions.

If the terminal 100 calculates a color difference, the terminal 100 may detect the first region and the second region set based on user input as described above from the captured image, and perform processing similar to that described above.

If the server 200 performs the processing, the terminal 100 may transmit the setting information of the first region and the second region set based on user input as described above to the server 200 together with data of the captured image, and the server 200 may detect the first region and the second region contained in the received setting information, from the captured image, and perform processing similar to that described above.

### (5) Processing at Terminal

The processing is not limited to that, for example, to which a client-server system is applied as described above, and all of the above-described processing may be performed at the terminal 100. In this case, for example, the processing of the server 200 shown in FIG. 8 may be applied in a similar manner to the color information processing shown in FIG. 5 to which the configuration example of the terminal 100A shown in FIG. 4 is applied, and similar processing may be performed at the terminal 100A.

### (6) Color System

In the foregoing embodiment, color information expressed in hue, saturation, and lightness defined in the HSL color space was used as the color information, but there is no limitation to this.

Specifically, for example, color information expressed in YCbCr may be used. Also, color information expressed in RGB may be used. These color systems are in a mapping relationship. For example, YCbCr and RGB can be converted into each other through a linear transformation. A method as in the foregoing embodiment can be applied to any color system.

### (7) Terminal

The user's terminal 100 may be various devices such as cameras, PDAs, personal computers, navigation devices, wristwatches, and various tablet terminals, in addition to cell phones such as smartphones, as described above.

Furthermore, the user's terminal 100 does not necessarily have to include the image capturing unit 150. In this case, for example, the terminal 100 may acquire data of a captured image from an external apparatus including the image capturing unit 150, and perform the above-described image processing based on the acquired data of the captured image.

### (8) Recording Medium

In the foregoing embodiment, various programs and data related to image processing were stored in the storage units, and the processing units read and executed these programs to realize the image processing of the foregoing embodiment. In this case, the storage units of the apparatuses are not only internal storage devices such as ROM, EEPROM, flash memory, hard disk, or RAM, but also recording media (recording media, external storage devices, or storage media) such as memory cards (SD cards), Compact Flash (registered trademark) cards, memory sticks, USB memory, CD-RW (optical disk), or MO (magneto-optical disk), and the various programs and data mentioned above may be stored in these recording media.

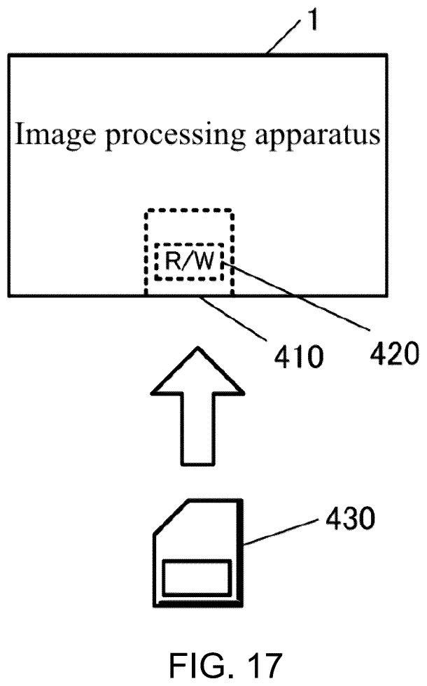

FIG. 17 is a diagram showing an example of a recording medium in this case.

In this example, the image processing apparatus 1 is provided with a card slot 410 into which a memory card 430 is to be inserted, and further provided with a card reader/writer (R/W) 420 configured to read information stored in the memory card 430 inserted in the card slot 410 or to write information to the memory card 430.

The card reader/writer 420 operates to write programs and data recorded in the storage unit to the memory card 430 in accordance with the control of the processing unit. The programs and data recorded in the memory card 430 are configured to be read by an external apparatus other than the image processing apparatus 1, so that image processing in the foregoing embodiment can be realized in the external apparatus.

The above-described recording medium can also be applied to various devices such as terminals, servers, electronic apparatuses (electronic devices), color information analyzers, and information processing apparatuses described in the foregoing example, for example.

### Others

In the foregoing example, the image processing apparatus 1 may be configured as an apparatus such as a skin analyzer. In addition, an apparatus for making a notification regarding rough skin may be configured as an apparatus including the above-described image processing apparatus 1 and a notification unit.

The technique of calculating color relative relationship information from first color information and second color information of an image in which the subject is a human and displaying the color relative relationship information in time series can be applied to various monitoring methods. According to an example, the technique described above can be applied as a non-contact vital sensing technique for measuring the fatigue level of a worker. A worker is, without limitation, a person who is engaged in work in which overexertion may lead to accidents. Such workers are, for example, drivers of large vehicles.

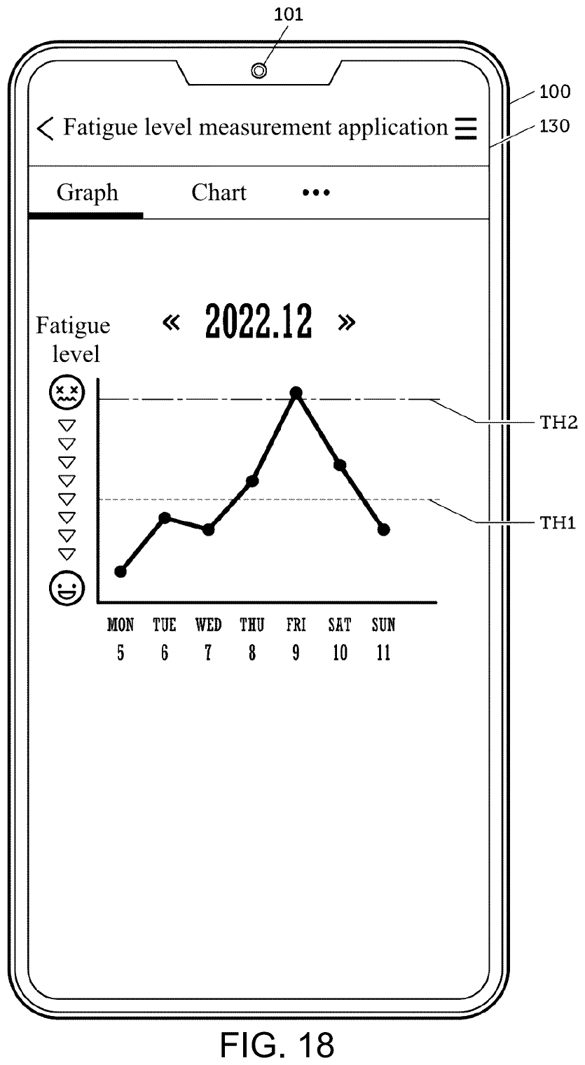

FIG. 18 is a diagram showing a screen example of a fatigue level measurement application. According to an example, the terminal 100 is a smartphone and has an in-camera 101 and the display unit 130. A worker captures an image of himself/herself with the in-camera 101 every day, for example. For a plurality of images captured on different days, color relative relationship information is calculated in the manner described above. The calculated color relative relationship information is displayed in time series according to the operation performed by the terminal operator. The example in FIG. 18 illustrates the transition of the color relative relationship information from Dec. 5, 2022 to Dec. 11, 2022. The color relative relationship information is a color difference or a color relative relationship vector, and the larger the value is, the higher the fatigue level that the terminal operator is informed of is, and the smaller the value is, the lower the fatigue level is. In this example, a first threshold value TH1 is set as the fatigue level at which a certain level of fatigue is recognized, and a second threshold value TH2 is set as the fatigue level at which fatigue has reached a serious degree. The number of threshold values may be one, or a plurality of threshold values may be set in a stepwise manner. In the example in FIG. 18, the fatigue level exceeding the first threshold value TH1 is detected on December 8, the fatigue level exceeding the second threshold value TH2 is detected on December 9, and the threshold value exceeding the first threshold value TH1 is detected on December 10. According to an example, when the fatigue level exceeding the threshold value is detected, a notification may be made to an external administrator or the like using the communication function of the terminal 100, a message urging the user to take a rest because the fatigue level is increasing may be displayed on the display unit 130, or the message may be read out by voice.

According to another example, the above-described technique can be used to confirm the effectiveness of esthetics, gyms, health foods, and the like. According to another example, the above-described technique can be used as one of the detection conditions to confirm the intention of dementia patients or the like.

The function of generating time-series data of color relative relationship information can be realized by a processing circuit. That is to say, the processing circuit calculates color information, calculates color relative relationship information, and generates time-series data of color relative relationship information. The processing circuit may be dedicated hardware or a CPU (Central Processing Unit, also called a central processing circuit, processing device, arithmetic circuit, microprocessor, microcomputer, or DSP) that executes a program stored in a memory.

If the processing circuit is dedicated hardware, the processing circuit may be, for example, a single circuit, a complex circuit, a programmed processor, a parallel programmed processor, an ASIC, an FPGA, or a combination thereof.

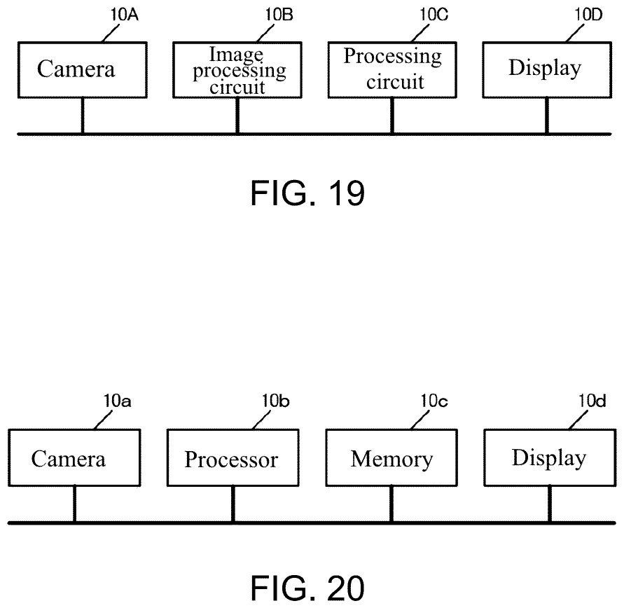

FIG. 19 is a diagram showing a configuration example of a terminal. An image processing circuit 10B generates JPEG and other data from data captured by a camera 10A. The image data generated by the image processing circuit 10B is provided to a processing circuit 10C. The processing circuit 10C generates the above-described time-series data of color relative relationship information. The time-series data is then displayed on a display 10D in response to an operation performed by a terminal user.

FIG. 20 shows a configuration example in the case in which the processing circuit is a CPU. In this case, each function of the processing circuit is realized by software or a combination of software and firmware. The software or firmware is written as a program and stored in a memory 10c. A processor 10b reads and executes the program stored in the memory 10c to realize each function. In other words, this configuration includes the memory 10c configured to store programs that, when executed by the processing circuit in FIG. 20, result in the generation of the above-described time-series data of color relative relationship information. These programs can be said to cause the computer to execute the procedures and methods of FIGS. 3, 5, 8, etc. In this example, the memory can be a non-volatile or volatile semiconductor memory, such as RAM, ROM, flash memory, EPROM, or EEPROM, a magnetic disk, a flexible disk, an optical disk, a compact disk, a mini disk, a DVD, or the like. It will be appreciated that some of the above-described functions may be realized by hardware and some by software or firmware.

### LIST OF REFERENCE NUMERALS

1 Image processing apparatus
100 Terminal
200 Server
300 Network

1. An image processing apparatus comprising:
a processing circuit configured to calculate relative relationship information on a difference between first color information in a first region included in a captured image and second color information in a second region included in the captured image; and
a display configured to display the relative relationship information in time-series of a plurality of captured images captured at different points in time,
wherein the first region and the second region are different regions of a same person.

2. The image processing apparatus according to claim 1, wherein the relative relationship information contains information on a color difference between the first color information and the second color information.

3. (canceled)

4. The image processing apparatus according to claim 1, wherein the first region is a face region, a cheek region, or a neck region, and the second region is an inner region of an upper arm or an inner region of a wrist.

5. The image processing apparatus according to claim 1, wherein the processing circuit calculates the relative relationship information from a plurality of captured images.

6-11. (canceled)

12. The image processing apparatus according to claim 1, the display configured to display temperature or humidity in which the images were captured, in addition to the time-series relative relationship information.
